SEQUENCE LISTING (52 sequences)

SEQ ID NO: 1 - DNA, 45 bp, Artificial Sequence
  Forward primer
gcataatactaccagtctcctcagcaagctacgcattttcattag

SEQ ID NO: 2 - DNA, 44 bp, Artificial Sequence
  Reverse primer
tagaatagtcgcatacttcctcagccataacatctcctcgaact

SEQ ID NO: 3 - DNA, 18 bp, Artificial Sequence
  Probe
ntaattcatcaacaatgn

SEQ ID NO: 4 - DNA, 19 bp, Artificial Sequence
  Forward bumper
aggtaatggtgcagtaggt

SEQ ID NO: 5 - DNA, 17 bp, Artificial Sequence
  Reverse bumper
ccagctttcacacgaac

SEQ ID NO: 6 - DNA, 20 bp, Artificial Sequence
  pDNA capture probe
nagtgtctaaatcaatgatg

SEQ ID NO: 7 - DNA, 18 bp, Artificial Sequence
  Biotinilated detection probe
ctaattcatcaacaatgn

SEQ ID NO: 8 - DNA, 44 bp, Artificial Sequence
  Design 1 forward primer
gaaacaatgtacctgtcacctcagcgaccgaaacaatgtggaat

SEQ ID NO: 9 - DNA, 44 bp, Artificial Sequence
  Design 1 reverse primer
ttcaatagtcagttacttcctcagcggaacgatgcctaatctca

SEQ ID NO: 10 - DNA, 17 bp, Artificial Sequence
  Design 1 probe
ncaatacaggaacacan

SEQ ID NO: 11 - DNA, 23 bp, Artificial Sequence
  Design 1 forward bumper
gaaaatttaaaatcagaacgtgg

SEQ ID NO: 12 - DNA, 23 bp, Artificial Sequence
  Design 1 reverse bumper
gctttntaatcttttttagatac

SEQ ID NO: 13 - DNA, 14 bp, Artificial Sequence
  Design 1 pDNA capture probe
naatgtggnattgg

SEQ ID NO: 14 - DNA, 17 bp, Artificial Sequence
  Design 1 biotinilated detection probe
ccaatacaggaacacan

SEQ ID NO: 15 - DNA, 45 bp, Artificial Sequence
  Design 2 forward primer
ccattatactacctgtctcctcagcggcaaagatattcaactaac

SEQ ID NO: 16 - DNA, 47 bp, Artificial Sequence
  Design 2 reverse primer
tagaatagtcagttacttcctcagcgccataatcatttttcatgttg

SEQ ID NO: 17 - DNA, 18 bp, Artificial Sequence
  Design 2 probe
nttttgaactttagcatn

SEQ ID NO: 18 - DNA, 23 bp, Artificial Sequence
  Design 2 forward bumper
gataatagcaatacaatcgcaca

SEQ ID NO: 19 - DNA, 23 bp, Artificial Sequence
  Design 2 reverse bumper
gtgctaataattcacctgtttga

SEQ ID NO: 20 - DNA, 19 bp, Artificial Sequence
  Design 2 pDNA capture probe
ntttagcatcaatagttag

SEQ ID NO: 21 - DNA, 19 bp, Artificial Sequence
  Design 2 biotinilated detection probe
gttntaaatnctcttttgn

SEQ ID NO: 22 - DNA, 40 bp, Artificial Sequence
  Design 3 limiting primer L1
aataaatcataaggatcaacgtgttataggttctggtaca

SEQ ID NO: 23 - DNA, 36 bp, Artificial Sequence
  Design 3 excess primer E1
aataaatcataaggatctgagcatcgacgctacgtg

SEQ ID NO: 24 - DNA, 16 bp, Artificial Sequence
  Design 3 forward bumper 1
atggaaattctctggt

SEQ ID NO: 25 - DNA, 15 bp, Artificial Sequence
  Design 3 reverse bumper 1
tgtcaccatgttcac

SEQ ID NO: 26 - DNA, 40 bp, Artificial Sequence
  Design 4 limiting primer L2
aataaatcataaggatctggtgaacatggtgacactgaat

SEQ ID NO: 27 - DNA, 38 bp, Artificial Sequence
  Design 4 excess primer E2
aataaatcataaggatcgccctcaggacgttgttcaag

SEQ ID NO: 28 - DNA, 14 bp, Artificial Sequence
  Design 4 forward bumper 2
agcgtcgatgctca

SEQ ID NO: 29 - DNA, 17 bp, Artificial Sequence
  Design 4 reverse bumper 2
aatttgttcaatttgcg

SEQ ID NO: 30 - DNA, 42 bp, Artificial Sequence
  Table 4 limiting primer L1
ccaatatagtaacagtctcctcagcattcgcccttctgcacg

SEQ ID NO: 31 - DNA, 44 bp, Artificial Sequence
  Table 4 excess primer E1
ttcaaaagacccatacttcctcagccttctcattttttctaccg

SEQ ID NO: 32 - DNA, 17 bp, Artificial Sequence
  Table 4 forward bumper 1
tcggatccactagtaac

SEQ ID NO: 33 - DNA, 20 bp, Artificial Sequence
  Table 4 reverse bumper 1
gtgatggatatctgcagaat

SEQ ID NO: 34 - DNA, 16 bp, Artificial Sequence
  Table 4 chimeric pDNA/DNA
natcttgtaccaatgc

SEQ ID NO: 35 - DNA, 14 bp, Artificial Sequence
  Table 4 biotinilated probe
cgtggtccgtaaan

SEQ ID NO: 36 - DNA, 240 bp, Artificial Sequence
  Table 4 IC2
tttcacacaggaaacagctatgaccatgattacgccaagctatttaggtgacactatagaatactcaagctatgcatcaagcttggtaccgagctcggatccactagtaacggccgccagtgtgctggaattcgcccttctgcacggaccagttactttacggaccacgtaccgcattggtacaagatctccggtagaaaaaatgagaagggcgaattctgcagatatccatcacactgg

SEQ ID NO: 37 - DNA, 48 bp, Artificial Sequence
  Table 5 limiting primer L1
gcattatagtacctgtctcctcagctggtgaacatggtgacactgaat

SEQ ID NO: 38 - DNA, 46 bp, Artificial Sequence
  Table 5 excess primer E1
ttgaatagtcggttacttcctcagcgccctcaggacgttgttcaag

SEQ ID NO: 39 - DNA, 14 bp, Artificial Sequence
  Table 5 forward bumper 1
agcgtcgatgctca

SEQ ID NO: 40 - DNA, 17 bp, Artificial Sequence
  Table 5 reverse bumper 1
aatttgttcaatttgcg

SEQ ID NO: 41 - DNA, 44 bp, Artificial Sequence
  Table 6 limiting primer L1
gcattatagtacctgtctcctcagcgaattccctgcatcaatac

SEQ ID NO: 42 - DNA, 43 bp, Artificial Sequence
  Table 6 excess primer E1
gcattatggtacctctctcctcagctntgtcnatatcnncatc

SEQ ID NO: 43 - DNA, 21 bp, Artificial Sequence
  Table 6 forward bumper 1
aactaaggccaaagcttatac

SEQ ID NO: 44 - DNA, 19 bp, Artificial Sequence
  Table 6 reverse bumper 1
cagtcagtagtagaccatg

SEQ ID NO: 45 - DNA, 19 bp, Artificial Sequence
  Table 6 chimeric pDNA/DNA
ntacaaattatcactttga

SEQ ID NO: 46 - DNA, 17 bp, Artificial Sequence
  Table 6 biotinilated probe
tnatcgcatattaacan

SEQ ID NO: 47 - DNA, 17 bp, Artificial Sequence
  Table 6 FAM probe
naatcgcatannaacan

SEQ ID NO: 48 - DNA, 48 bp, Artificial Sequence
  Table 7 limiting primer L1
gaatagacccatacatcctcagcgacttgagtaatgataaattgatag

SEQ ID NO: 49 - DNA, 48 bp, Artificial Sequence; identical to SEQ ID NO: 48
  Table 7 excess primer E1

SEQ ID NO: 50 - DNA, 21 bp, Artificial Sequence
  Table 7 forward bumper 1
ccncttgcttataactgtatg

SEQ ID NO: 51 - DNA, 20 bp, Artificial Sequence
  Table 7 reverse bumper 1
gtttccntagaaaccttcat

SEQ ID NO: 52 - DNA, 17 bp, Artificial Sequence
  Table 7 FAM probe
nttgattccgttttgan

(19) United States
(12) Patent Application Publication
(10) Pub. No.: US 2014/0255928 A1
(43) Pub. Date: Sep. 11, 2014

(54) METHODS FOR TRUE ISOTHERMAL STRAND DISPLACEMENT AMPLIFICATION

(71) Applicant: Elitech Holding B.V., Spankeren (NL)

(72) Inventors: Yevgeniy S. Belousov, Mill Creek, WA (US); Boris Alabeyev, Lynnwood, WA (US); Noah Scarr, Seattle, WA (US)

(73) Assignee: Elitech Holding B.V., Spankeren (NL)

(21) Appl. No.: 14/202,637

(22) Filed: Mar. 10, 2014

Related U.S. Application Data
(60) Provisional application No. 61/776,256, filed on Mar. 11, 2013.

(51) Int. Cl. C12Q 1/68

(52) U.S. Cl. CPC C12Q 1/6844
USPC 435/6.11; 435/6.12

(57) ABSTRACT

Methods, primers and probes are provided for the isothermal amplification and detection, without denaturation, of double stranded nucleic acid targets for polymerase strand displacement amplification (“iSDA”). The methods and compositions disclosed are highly specific for nucleic acid targets with high sensitivity, specificity and speed that allow detection of clinical relevant target levels. The methods and compositions can easily be used to amplify or detect nucleic acid targets in biological samples.

This application claims priority to U.S. Provisional Patent Application Ser. No. 61/776,256, filed Mar. 11, 2013, entitled “Methods for True Isothermal Strand Displacement Amplification,” the entire contents of which are hereby incorporated by reference.

### BACKGROUND

This disclosure pertains to methods for isothermal strand displacement amplification that accomplishes efficient primer extension amplification with target specific primers and does not require pre-denaturation.

Isothermal amplification requires single stranded targets for efficient primer extension. Helicase dependent amplification of nucleic acids also requires helicase enzyme for unwinding double strands to allow amplification with a DNA polymerase (U.S. Pat. No. 7,282,328). Exponential strand displacement amplification (“SDA”) as described in U.S. Pat. No. 5,455,166 requires an initial denaturation of the target into single-stranded DNA (ssDNA), generation of hemiphosphorothioate sites which allow single strand nicking by restriction enzymes, and extension by a polymerase lacking 5′-3′ exonuclease activity. Raising the temperature of the reaction to approximately 95° C. to render double strands into single strands is required to permit binding of the primers to the target strands. State of the art SDA amplification requires the denaturation of the target at elevated temperature to yield ssDNA for strand displacement isothermal amplification.

The use of a nicking enzyme to cleave one of the strands of a target instead of the generation of hemiphosphorothioate sites in SDA amplification was described in (Ehses et al, J. Biochem. Biophys. Methods. 63:170-86 (2005)). The design of primers to reduce non-predictable byproducts was also described. Denaturation at 95° C. was required by Ehses et al. after the addition of target and before the addition of any enzymes. Nicking enzyme SDA amplification without denaturation of target at 95° C. was reported in U.S. Patent Application Publication No. 2009/0092967. However, a limitation of the latter method is that a limited number of nicking enzymes are available and quite often no natural nicking site is present in a target region of interest. An abasic site endonuclease amplification assay was disclosed in U.S. Patent Application Publication No. 2004/0101893. The use of this assay as a post amplification detection system in combination with other amplification systems was also disclosed. These assays require a denaturation step of dsDNA.

It is known in the art that double stranded (ds) nucleic acid can be denatured in different ways. Heat denaturation is state of the art to separate ds DNA into single strands. Native DNA denatures at about 85° C. (White, Handler and Smith, Principles of Biochemistry 5^{th} Edition, McGraw-Hill Kogakush, Ltd, pages 192-197, 1993). Early on, it was established that primer extension in amplification required the binding of a primer to a single strand DNA strand. This was preferably achieved by heating the sample at about 95° C. (M Panaccio and A Lew. PCR based diagnosis in the presence of 8% (v/v) blood. Nucleic Acids Res., 19: 1151 (1991)). It was recently reported that Watson-Crick pairs in naked DNA spontaneously flip into Hoogstein pairs under ordinary conditions, suggesting that DNA breathes (Fran-Kamentskii. Artificial DNA; PNA & XNA, 2:1, 1-3 (2011)).

A few nucleases cut just one strand of DNA thereby introducing a nick into DNA (Besnier and Kong, EMBO Reports, 21: 782-786 (2001)). Most such proteins are involved in DNA repair and other DNA-related metabolism and cannot easily be used to manipulate DNA. They usually recognize long sequences and associate with other proteins to form active complexes that are difficult to manufacture (Higashitani et al., J. Mol. Biol., 237: 388-4000 (1994)). Single strand nicking endonucleases which nick only one strand of the DNA double strands and traditional restriction endonucleases are listed and updated in the REBASE Database (rebase.neb.com; Roberts et al., Nucl. Acids Res., 31: 418-420 (2003)). Engineering of a nicking endonuclease has been described (Xu et al, PNAS 98: 12990-12995 (2001)).

Other methods using isothermal amplification have been disclosed recently (Niemz et al., Trends in Biotechnol., 29:240-250 (2011)). However, these amplification methods also utilize thermal or other denaturation.

### SUMMARY

The present invention relates generally to an isothermal assay which utilizes the advantages of target nucleic acid amplification without the requirement for dsDNA denaturation. The present methods enable efficient detection of target nucleic acids with exquisite specific amplification. The present disclosure unexpectedly determined that primers designed according to a particular method allow efficient primer extension amplification of target specific primers without pre-denaturation. Generally, the present disclosure provides methods, primers and probes for the isothermal amplification without denaturation of nucleic acid targets for polymerase primer extension (isothermal strand displacement amplification (“iSDA”)) in samples including biological samples (e.g., blood, nasopharyngeal or throat, swab, wound swab, or other tissues). The nucleic acid targets may be double stranded or they may be single stranded, such as RSV virus. RNA targets may be single stranded or double stranded.

The method described herein utilizes primer oligonucleotides that allow primer extension without denaturation of nucleic acid targets. In some examples the primers have modified bases to improve stability or to eliminate primer self-association. In one embodiment modified bases are used to limit primer self-association.

In certain examples the primer comprises a 5′-non-complementary tail wherein said tail further comprises a nicking enzyme specific sequence.

In the methods described herein, the nucleic acids present in a clinical or test sample obtained from a biological sample or tissue suspected of containing a clinical target (microorganisms or tissue, for example) are extracted with methods known in the art. The target nucleic acids are amplified without denaturation and detected. More specifically the target specific primers contain a sequence specific for target and a non-target complementary 5′-tail, wherein the tail contains a sequence specific for a nicking enzyme when hybridized to its complementary sequence. At least one amplification cycle provides a double stranded amplicon containing a nicking site which allows strand displacement in a second amplification cycle. The amplified nucleic acid can be detected by a variety of state of the art methods including fluorescence resonance energy (“FRET”), radiolabels, lateral flow, enzyme labels, and the like.

The methods described herein also include methods for the design of primers allowing amplification of at least one cycle of amplification without denaturation of duplex DNA target.

In certain methods provided herein the methods comprise the detection of iSDA or RT-iSDA amplified targets by lateral flow.

Those skilled in the art will appreciate that the present disclosed amplification method can be performed in combination with other methods. In one embodiment the amplification method described in U.S. Patent Application Publication No. 2009/0092967 can be combined with the method of the present disclosure.

This disclosure provides an isothermal method for specifically detecting a nucleic acid sequence in a biological sample from an individual. The disclosure also provides oligonucleotide primers and probes comprising nucleotide sequences characteristic of specific genomic nucleic acid sequences. The method includes performing isothermal amplification without a denaturation step prior to amplification. The amplification step includes contacting the sample nucleic acid with pairs of primers to produce amplification product(s) if the specific genomic nucleic acid target is present. The preferred primers target a specific region of a specific target gene. Each of the preferred primers has a 5′-oligonucleotide tail non-complementary to the target where said non-complementary tail contains a sequence when hybridized to a complementary sequence contains a nicking enzyme cleavage site. The oligonucleotide probes detect the amplified target directly or indirectly. The preferred oligonucleotide probe is a 5′-minor groove binder-fluorophore-oligonucleotide-quencher-3′ conjugate that fluoresces on hybridization to its complementary amplified target. In some embodiments one or more primer is labeled. In some embodiments a double strand binding fluorescent dye is used. In some embodiments one or more bumper oligonucleotides are provided. In some embodiments the probe(s) is omitted. In some embodiments the amplified target is captured on a solid support or membrane and detected by a labeled probe. In some embodiments the primer concentrations are present in different concentrations. In some embodiments an internal control is provided.

### BRIEF DESCRIPTION OF THE DRAWINGS

FIG. 1 shows a schematic of an example of dual capture and detection of iSDA amplified amplicon by pDNA immobilized on a solid surface;

FIG. 2 shows an example of real-time iSDA amplification of different concentrations of the ldh1 gene with fluorescence detection utilizing a Pleiades probe;

FIG. 3 shows an example of lateral flow colorimetric detection of an ldh1 iSDA amplified amplicon with the approach provided in FIG. 1;

FIG. 4 shows an example of real-time iSDA amplification of two different mecA designed assays with fluorescence detection utilizing a Pleiades probe;

FIG. 5 shows an example of real-time iSDA amplification with different polymerases;

FIG. 6 shows an example of gel analysis of the valuation of Nt.Alw I on PCR Amplified target containing Nt.Alw I cleavage site;

FIG. 7 shows an example of lateral flow detection of iSDA biplex-amplified ldh1 and IC amplicons;

FIG. 8 shows a schematic representation of a primer containing a complementary- and non-complementary-sequence;

FIG. 9 shows the probe specific iSDA detection and differentiation of ldh1 gene in S. aureus and of S. epidermis;

FIG. 10 shows the specific real-time iSDA amplification of S. aureus nucleic acid extracted with five different extraction methods;

FIG. 11 shows the results of amplification reactions comparing amplification with primers and probes optimized for use in the present isothermal strand displacement amplification method and traditional primers and probes;

FIG. 12 shows the specific reverse transcriptase-iSDA (RT-iSDA) amplification of Respiratory syncytial virus (RSV) extracted RNA nucleic acid using both real-time fluorescence detection and post-amplification lateral flow detection; and

FIG. 13 shows the real-time iSDA amplification of native and denatured Plasmodium falciparum DNA.

### DETAILED DESCRIPTION OF PREFERRED EMBODIMENTS

### I. General

Generally, the present disclosure provides methods, primers and probes for the isothermal amplification and detection, without denaturation, of double stranded nucleic acid targets for polymerase strand displacement amplification (“iSDA”). The methods and compositions disclosed are highly specific for nucleic acid targets with high sensitivity, specificity and speed that allow detection of clinical relevant target levels. The methods and compositions can easily be used to amplify or detect nucleic acid targets in biological samples.

According to Ehses et al. (J. Biochem. Biophys. Methods. 63:170-86 (2005), incorporated herein by reference), primers can be designed using the Vienna Folding Package (tbi.univie.ac.at/ivo/RNA/) that identifies analyzes sequences that allowing one to minimize the accumulation of non-predictable byproducts especially for longer incubation times and low concentrations of initial template DNA. More specifically, the Vienna Folding Package is a software product that predicts a secondary structure of the primers based on the calculations of the minimum free energy of the hybridization reaction and calculates the probabilities of alternative DNA/DNA duplex structures. Primers designed using software such as the Vienna Folding Package are considered to have an improved hybridization stringency, and thus permit efficient elongation of a target sequence. The Tₘ of the selected primers can then be adjusted by calculation with a preferred software package, such as the Eclipse Design Software 2.3 (Afonina et al., Single Nucleotide Polymorphism Detection with fluorescent MGB Eclipse Systems in A-Z of Quantitative PCR, Ed. S. A. Bustin, International University Line, La Jolla, Calif. pages 718-731 and XII-XIII, 2004; see also U.S. Pat. Nos. 6,683,173 and 7,751,982). The software adjusts the Tm of the primers for optimum extension as well, by calculating duplex stabilities using an algorithm applying a nearest-neighbor model for duplex formation thermodynamics for each of the neighboring base pairs. Each nearest neighbor thermodynamic parameter defines a thermodynamic contribution of two corresponding neighboring bases. A preferred oligonucleotide primer sequence is then selected having the desired duplex stability. The primers can also be designed, if necessary or desired, to include modified bases (see U.S. Pat. No. 7,045,610; U.S. Pat. No. 6,127,121; U.S. Pat. No. 6,660,845; U.S. Pat. No. 5,912,340 and US Application Publication No. 2010/057862, all incorporated by reference). In the case of probes or MGB probes, the same software package (such as Eclipse Design Software 2.3) can be used.

### II. Definitions

A “sample” as used herein refers to a sample of any source which is suspected of containing a target sequence. These samples can be tested by the methods described herein. A sample can be from a laboratory source or from a non-laboratory source. A sample may be suspended or dissolved in liquid materials such as buffers, extractants, solvents, and the like. Samples also include biological samples such as plant, animal and human tissue or fluids such as whole blood, blood fractions, serum, plasma, cerebrospinal fluid, lymph fluids, milk, urine, various external secretions of the respiratory, intestinal, and genitourinary tracts, tears, and saliva; and biological fluids such as cell extracts, cell culture supernatants, fixed tissue specimens, and fixed cell specimens. Samples include nasopharyngeal or throat swabs, stools, wound or rectal swabs. Biological samples may also include sections of tissues such as biopsy and autopsy samples or frozen sections taken for histological purposes. A biological sample is obtained from any animal including, e.g., a human. A biological sample may include human and animal pathogens that includes microbes or microorganisms such as a viruses, bacteria, or fungi that causes disease in humans. Biological samples may further also include products of gene mutated-metabolic disorders.

The terms “flap primer” or “overhang primer” refer to a primer comprising a 5′ sequence segment non-complementary to a target nucleic acid sequence, wherein said tail further comprises a nicking enzyme specific sequence and a 3′ sequence segment complementary to the target nucleic acid sequence The flap primers are suitable for primer extension or amplification of the target nucleic acid sequence The primers may comprise one or more non-complementary or modified nucleotides (e.g., pyrazolopyrimidines as described in U.S. Pat. No. 7,045,610 which is incorporated herein by reference) at any position including. e.g., the 5″ end.

The term “isothermal strand displacement amplification” (“iSDA”) refers to primer extension using a primer that comprises a 5′ sequence segment non-complementary to a target nucleic acid sequence, wherein said tail may further comprise a nicking enzyme specific sequence and a 3′ sequence segment complementary to the target nucleic acid sequence.

The term “fluorescent generation probe” refers either to a) an oligonucleotide having an attached minor groove binder, fluorophore, and quencher, b) an oligonucleotide having an attached fluorophore, and quencher, c) an oligonucleotide having an attached minor groove binder, and fluorophore, d) an oligonucleotide having an attached fluorophore and quencher, e) an oligonucleotide having an attached fluorophore, or f) a DNA binding reagent. The probes may comprise one or more non-complementary or modified nucleotides (e.g., pyrazolopyrimidines as described in U.S. Pat. No. 7,045,610) at any position including, e.g., the 5′ end. In some embodiments, the fluorophore is attached to the modified nucleotide. In some embodiments the probe is cleaved to yield a fluorescent signal.

Preferably, modified bases increase thermal stability of the probe-target duplex in comparison with probes comprised of only natural bases (i.e., increase the hybridization melting temperature of the probe duplexed with a target sequence). Modified bases can decrease probe and primer self-association compared to only normal bases. Modified bases include naturally-occurring and synthetic modifications and analogues of the major bases such as, for example, hypoxanthine, 2-aminoadenine, 2-thiouracil, 2-thiothymine, inosine, 5-N⁴-ethenocytosine, 4-aminopyrrazolo[3,4-d]pyrimidine and 6-amino-4-hydroxy-[3,4-d]pyrimidine. Any modified nucleotide or nucleotide analogue compatible with hybridization of probe with a nucleic acid conjugate to a target sequence is useful, even if the modified nucleotide or nucleotide analogue itself does not participate in base-pairing, or has altered base-pairing properties compared to naturally-occurring nucleotides. Examples of modified bases are disclosed in U.S. Pat. Nos. 7,045,610; 5,824,796; 6,127,121; 5,912,340; and PCT Publications WO 01/38584; WO 01/64958, each of which is hereby incorporated herein by reference in its entirety. Preferred modified bases include 5-hydroxybutynyl uridine for uridine; 4-(4,6-Diamino-¹H-pyrazolo[3,4-d]pyrimidin-3-yl)-but-3-yn-1-ol, 4-amino-¹H-pyrazolo[3,4-d]pyrimidine, and 4-amino-¹H-pyrazolo[3,4-d]pyrimidine for adenine; 5-(4-Hydroxy-but-1-ynyl)-1H-pyrimidine-2,4-dione for thymine; and 6-amino-¹H-pyrazolo[3,4-d]pyrimidin-4(5H)-one for guanine. Particularly preferred modified bases are “Super A®: 4-(4,6-Diamino-1H-pyrazolo[3,4-d]pyrimidin-3-yl)-but-3-yn-1-ol,” “Super G®: 4-hydroxy-6-amino pyrazolopyrimidine” (www.elitechgroup.com) and “Super T®: 5-(4-hydroxy-but-1-ynyl)-1H-pyrimidine-2,4-dione”, “Super-D™: 3-Alkynyl pyrazolopyrimidine” analogues as universal bases are disclosed in U.S. Patent Application Publication No. 2012/0244535, incorporated by reference.

The terms “fluorescent label” or “fluorophore” refer to compounds with a fluorescent emission maximum between about 400 and about 900 nm. These compounds include, with their emission maxima in nm in brackets, Cy2™ (506), GFP (Red Shifted) (507), YO-PRO™-1 (509), YOYO™-1 (509), Calcein (517), FITC (518), Fluor X™ (519), Alexa™ (520), Rhodamine 110 (520), 5-FAM (522), Oregon Green™ 500 (522), Oregon Green™ 488 (524), RiboGreen™ (525), Rhodamine Green™ (527), Rhodamine 123 (529), Magnesium Cireen™ (531), Calcium Green™ (533), TO-PRO™-1 (533), TOTOR-1 (533), JOE (548), BODIPYR 530/550 (550), Dil (565), BODIPY® 558/568 (568), BODIPYR 564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAIVIRA (582), Rhodamine Red™ (590), Cy3.5™ (596), ROX (608), Calcium Crimson™ (615), Alexa™ 594 (615), Texas Red® (615), Nile Red (628), YO-PRO™-3 (631), YOYO™-3 (631), R-phycocyanin (642), C-Phycocyanin (648), TO-PRO™-3 (660), TOTO®-3 (660), DiD DilC(5) (665), Cy5™ (670), Thiadicarbocyanine (671), and Cy5.5 (694). Additional fluorophores are disclosed in PCT Patent Publication No. WO 03/023357 and U.S. Pat. No. 7,671,218. Examples of these and other suitable dye classes can be found in Haugland et al., Handbook of Fluorescent Probes and Research Chemicals, Sixth Ed., Molecular Probes, Eugene, Ore. (1996); U.S. Pat. Nos. 3,194,805; 3,128,179; 5,187,288; 5,188,934; 5,227,487, 5,248,782; 5,304,645; 5,433,896; 5,442,045; 5,556,959; 5,583,236; 5,808,044; 5,852,191; 5,986,086; 6,020,481; 6,162,931; 6,180,295; and 6,221,604; EP Patent No. 1408366; Smith et al., J. Chem. Soc. Perkin Trans. 2:1195-1204 (1993); Whitaker et al., Anal. Biochem. 207:267-279 (1992); Krasoviskii and Bolotin, Organic Luminescent Materials, VCH Publishers, NY. (1988); Zolliger, Color Chemistry, 2nd Edition, VCH Publishers, NY. (1991); Hirschberg et al., Biochemistry 37:10381-10385 (1998); Fieser and Fieser, REAGENTS FOR ORGANIC SYNTHESIS, Volumes 1 to 17, Wiley, US (1995); and Geiger et al., Nature 359:859-861 (1992). Still other dyes are provided via online sites such as www.zeiss.com. Phosphonate dyes are disclosed in co-owned U.S. Pat. No. 7,671,218 and U.S. Pat. No. 7,767,834.

There is extensive guidance in the art for selecting quencher and fluorophore pairs and their attachment to oligonucleotides (Haugland, 1996; U.S. Pat. Nos. 3,996,345 and 4,351,760 and the like). Preferred quenchers are described in U.S. Pat. No. 6,727,356, incorporated herein by reference. Other quenchers include bis azo quenchers (U.S. Pat. No. 6,790,945) and dyes from Biosearch Technologies, Inc. (provided as Black Hole™ Quenchers: BH-1, BH-2 and BH-3 quenchers), Dabcyl, TAMRA and carboxytetramethyl rhodamine.

The term “linker” refers to a moiety that is used to assemble various portions of the molecule or to covalently attach the molecule (or portions thereof) to a solid support, surface or membrane. Typically, a linker or linking group has functional groups that are used to interact with and form covalent bonds with functional groups in the ligands or components (e.g., fluorophores, oligonucleotides, minor groove binders, or quenchers) of the conjugates described and used herein. Examples of functional groups on the linking groups (prior to interaction with other components) include —NH2, —NHNH₂, —ONH₂, —NHC═(O)NHNH₂, —OH, and —SH. The linking groups are also those portions of the molecule that connect other groups (e.g., phosphoramidite moieties and the like) to the conjugate. Additionally, a linker can include linear or acyclic portions, cyclic portions, aromatic rings, and combinations thereof.

The term “solid support” refers to any support that is compatible with oligonucleotide attachment, including, for example, glass, controlled pore glass, polymeric materials, polystyrene, beads, coated glass, and the like.

Lateral flow assay technology is well known in the art and is performed on strips of porous paper or sintered polymer see for example U.S. Pat. No. 6,485,982, U.S. Pat. No. 7,799,554, and U.S. Pat. No. 7,901,623.

In the description herein, the abbreviations MGB, FL, Q, CPG, and ODM refer to “minor groove binder.” “fluorescent label” or “fluorophore,” “quencher,” “controlled pore glass” as an example of a solid support), and “oligonucleotide” moieties molecules, respectively, and in a manner which is apparent from context. The terms “probe” and “conjugate” are used interchangeably and refer to an oligonucleotide having an attached minor groove binder, fluorophore, and quencher.

The terms “oligonucleotide,” “nucleic acid,” and “polynucleotide” are used interchangeably herein. These terms refer to a compound comprising nucleic acid, nucleotide, or its polymer in either single- or double-stranded form, e.g., DNA, RNA, analogs of natural nucleotides, and hybrids thereof. The terms encompass polymers containing modified or non-naturally-occurring nucleotides, or to any other type of polymer capable of stable base-pairing to DNA or RNA including, but not limited to, peptide nucleic acids as described in Nielsen et al., Science, 254:1497-1500 (1991), bicyclo DNA oligomers as described in Bolli et al., Nucleic Acids Res., 24:4660-4667 (1996), and related structures. Unless otherwise limited, the terms encompass known analogs of natural nucleotides that hybridize to nucleic acids in a manner similar to naturally-occurring nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). A “subsequence” or “segment” refers to a sequence of nucleotides that comprise a part of a longer sequence of nucleotides. In some embodiments, nucleotides may include analogs of natural nucleotides which exhibit preferential binding to nucleotides other than naturally occurring DNA or RNA; an example of such nucleotides is pDNA (Eschenmoser et al, Helvetica Chimica Acta, “Why Pentose- and Hexose-Nucleic Acids?”, pp. 76: 2161-2183 (1993)).

The term “Nicking Enzyme (or nicking endonuclease)” describes an enzyme that cuts one strand of a double-stranded DNA at a specifically recognition recognized nucleotide sequences known as a nicking site. Such enzymes hydrolyse (cut) only one strand of the DNA duplex, to produce DNA molecules that are “nicked”, rather than cleaved. These nicking enzymes include N.Alw I, Nb.BbvCl, Nt.BbvCl, Nb.BsmI, Nt.BsmAI, Nt.BspQI, Nb.BsrDI, Nt.BstNBI, Nb.BstsCI, Nt.CviPII, Nb.Bpu10I, Nt.Bpu10I and Nt.Bst9I which are commercially available from www.neb.com, www.fermentas.com and www.sibenzyme.com, respectively. The New England Biolabs REBASE website (rebase.neb.com/cgi-bin/azlist?nick) lists 917 nicking enzymes. Designing of artificial nicking endonucleases on the basis of restriction endonucleases was reviewed by Zheleznaya et al., Biochemistry (Mosc). 74:1457-66 (2009), incorporated by reference. “Nicking Enzyme” also includes engineered enzymes that cut one strand of a double stranded DNA, for example, zinc finger nucleases.

The term “Lateral Flow” describes a porous membrane capable of nonabsorbent lateral flow used as assay substrate; a member of the binding pair is affixed in an indicator zone defined in the substrate. The sample is applied at a position distant from the indicator zone and permitted to flow laterally through the zone; any analyte in the sample is complexed by the immobilized specific binding member, and detected. Lateral flow utilizing immuno-binding pairs is well known in the art (U.S. Pat. No. 4,943,522). Lateral flow using DNA binding pairs was disclosed in U.S. Pat. No. 7,488,578. pDNA binding pairs are disclosed in co-owned US application 2012-0015358 A1. Biotin-streptavidin affinity pairs are well known in the art and commercially available. Streptavidin-coated label may be a covalent or adsorptively bound streptavidin or other biotin-binding species, and the label may be a polystyrene nanoparticle doped with fluorescent or visible dye, a carbon black nanoparticle, a metal colloid, or other species detectable by fluorescence, radioactivity, magnetism, or visual acumen. The lateral flow buffer may be an aqueous suspension containing detergents, proteins, surfactants, and salts. The lateral flow strip may be a porous matrix composed of nitrocellulose, modified nitrocellulose, polyethersulfone, cellulose, glass fiber, polyvinylidene fluoride, or nylon. The lateral flow strip has at least one detection region composed of affinity pairs specific to the iSDA reaction products.

The practice of the methods described herein will employ, unless otherwise indicated, conventional techniques in organic chemistry, biochemistry, oligonucleotide synthesis and modification, bioconjugate chemistry, nucleic acid hybridization, molecular biology, microbiology, genetics, recombinant DNA, and related fields as are within the skill of the art. These techniques are fully explained in the literature. See, for example, Sambrook, Fritsch & Maniatis, MOLECULAR CLONING: A LABORATORY MANUAL, Second Edition, Cold Spring Harbor Laboratory Press (1989); Ausubel, et al., CURRENT PROTOCOLS IN MOLECULAR BIOLOGY, John Wiley & Sons (1987, 1988, 1989, 1990, 1991, 1992, 1993, 1994, 1995, 1996); Gait (ed.), OLIGONUCLEOTIDE SYNTHESIS: A PRACTICAL APPROACH, IRL Press (1984); and Eckstein (ed.), OLIGONUCLEOTIDES AND ANALOGUES: A PRACTICAL APPROACH, IRL Press (199)).

### III. Descriptions

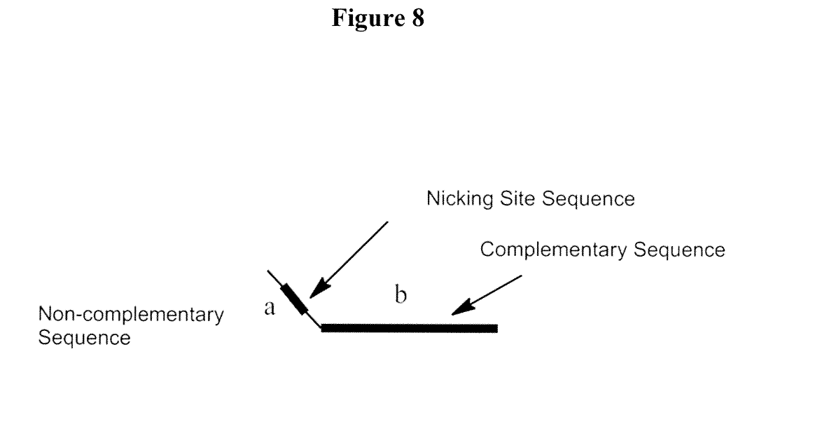

In one aspect, this disclosure provides an isothermal method for specifically detecting a nucleic acid sequence in a biological sample from an individual. The isothermal method can be carried out entirely at room temperature, or between about 40° C. and about 65° C., or more preferably between about 45° C. and about 55° C. The disclosure also provides oligonucleotide primers and probes comprising nucleotide sequences characteristic of a specific genomic nucleic acid sequences. The method includes performing of isothermal amplification without a denaturation step prior to amplification. The amplification step includes contacting the sample nucleic acid with pairs of primers to produce amplification product(s) if the specific genomic nucleic acid target is present. The primer “a-b” comprises a complementary sequence “b” and comprises a non-complementary nicking enzyme recognition sequence site “a” when hybridized to a complementary sequence (FIG. 8). Primer a-b further comprises sequences selected by free energy minimization for specific hybridization and efficient elongation. The primers target a specific region of a specific target gene that allows amplification without thermal denaturation. Bumper primers hybridize upstream of the 5″-end of the flap primers to generate a target specific single stranded DNA newly synthesized amplicon by strand displacement (Nuovo G J, .Diagn Mol. Pathol. 2000 December; 9(4):195-202.). The oligonucleotide probes detect the amplified target directly or indirectly. The preferred oligonucleotide probe is a 5′-minor groove binder-fluorophore-oligonucleotide-quencher-3′ conjugate that fluoresces on hybridization to its complementary amplified target.

In some embodiments the probe(s) is omitted. In some embodiments the amplified target is captured on a solid support, surface or membrane and detected by a labeled probe. In some embodiments the primer concentrations are present in different concentrations. In some embodiments an internal control is provided.

In a particular embodiment human, animal, and/or plant pathogen nucleic acids are amplified and detected.

In another embodiment the amplified target nucleic acid is RNA and the method further comprises a reverse transcriptase step.

In another aspect, the 5′ non-complementary sequence comprises a sequence for a nicking site. Although any enzyme with a suitable nicking site can be used, preferred nicking enzyme recognition sequences are selected from N.Alw I, Nb.BbvCl, Nt.BbvCl, Nb.BsmI, Nt.BsmAI, Nt.BspQI, Nb.BsrDI, Nt.BstNBI, Nb.BstsCI, Nb.Bpu10I, Nt.Bpu10I and Nt.Bst9I, Nb.Mva1269I and endo nuclease V.

In another embodiment, a complementary primer sequence comprises a sequence with an Endonuclease V (“Endo V”) cleavage site requiring no heat or chemical denaturation, as more fully described in U.S. Pat. No. 8,202,972 or U.S. Patent Application Publication No. 2011/0171649 incorporated by reference, which describes Endo V-based amplification primers. More specifically Endonuclease V is a repair enzyme recognizing DNA oligonucleotides containing deaminated modified bases such as inosine. Endo V cleaves the second or third phosphodiester bond 3′ to the modified base, such as inosine. U.S. Pat. No. 8,202,972 describes an Endonuclease V-based amplification method that extends a forward- and reverse-primer containing inosine adjacent to 3′-end terminal base. In the second round of amplification the Endo V cleaves the second or third phosphodiester bond 3′ to the inosine in the same strand. The 3′-hydroxyl of the nick is extended by DNA polymerase in a template-directed manner. Employing a series of nested primer pairs complementary upstream of the 5′-end of the inosine containing primer pair, a series of extension products are generated. U.S. Pat. No. 8,202,972 requires that “target dsDNA may be thermally denatured, chemically denatured, or both thermally and chemically denatured”.

### EXAMPLES

The following examples are provided to illustrate, but not to limit, the subject matter described herein.

In these examples, iSDA was performed using final concentrations of 3.75 mM MgSO₄, 50 mM KH₂PO₄ pH 7.6, 250 nM forward primer, 1 μM reverse primer, 50 nM bumper oligonucleotides, 200 nM probe, 0.2 mM dNTPs, 40 μg/mL BSA, 10 ng genomic DNA, 4U N.BbvC1B and 3.6U Bst DNA polymerase in a total volume of 20 μL (mono-reagent). Twenty microliters of the mono-reagent was introduced in a 96 well PCR plate with 10 μL of sample nucleic acid. Sample nucleic acid was obtained by extraction with easyMag using NucliSENSE easyMAG extraction reagents (Biomerieux, l'Etoile, France). The plate was sealed with MicroAmp® Optical Adhesive Film (Applied Biosystems, Foster City, Calif.) and then centrifuged to collect the assay solution in the bottom of the plate well. The assay was then performed in an ABI 7500 DX Fast Block Real-time PCR machine at 48° C. for 30 minutes.

### Example 1

This example demonstrates the efficient iSDA amplification without denaturation of the ldh1 gene from easyMag extracted nucleic acid from cultured S. aureus subsp. aureus COL (gi|57650036 :262250-263203). The primer, bumper and probe sequences are shown in Table 1.

Table 1 below illustrates ldh1 oligonucleotide sequences for iSDA amplification. Underlined sequences represents the nicking site for N.BbvC1B. The upper case sequence is ldh1 specific, the 5′-end lower case sequence is non-complementary to the ldh1 target, and the pDNA sequence is shown in brackets. Q14 is a hexaethylene glycol linker, MGB is a DPI₃ minor groove binder, FAM is fluorescein, and EDQ is the Eclipse® dark quencher (quenching range 390-625 nm, maximum absorption 522 nm, Epoch Biosciences, Inc., Bothell, Wash.).

TABLE 1

| Seq ID # | Description | Oligonucleotide sequence |
|---|---|---|
| 1 | Forward | gcataatactaccagtctcctcagc |
| | Primer | AAGCTACGCATTTTCATTAG |
| 2 | Reverse | tagaatagtcgcatacttcctcagc |
| | Primer | CATAACATCTCCTCGAACT |
| 3 | Probe | MGB-FAM-CTAATTCATCAACAATGC- |
| | | EDQ |
| 4 | Forward | AGGTAATGGTGCAGTAGGT |
| | Bumper | |
| 5 | Reverse | CCAGCTTTCACACGAAC |
| | Bumper | |
| 6 | pDNA | [TTTTTTTTC]-(Q14)-CAGTGTCTA |
| | Capture | AATCAATGATG |
| | Probe | |
| 7 | Biotinilated | CTAATTCATCAACAATGC-biotin |
| | Detection | |
| | Probe | |

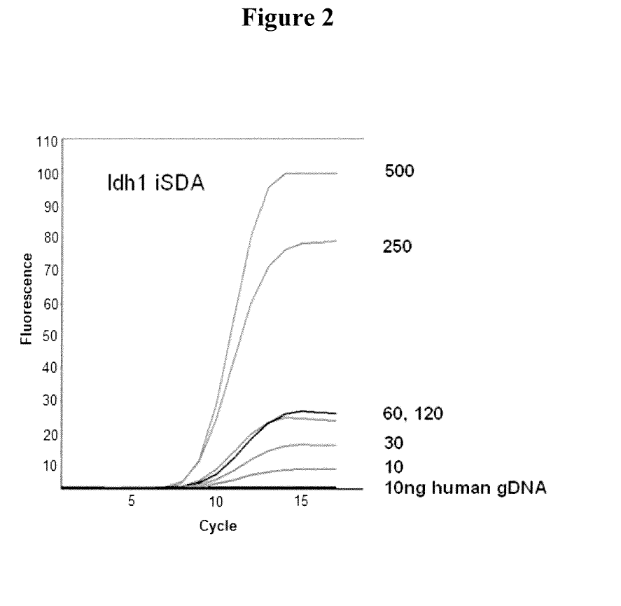

Real-time iSDA amplification with oligonucleotide 1 to 5 was performed as described above with target concentrations ranging from 10 to 500 copies per reaction. The results are shown in FIG. 2.

Lateral Flow:

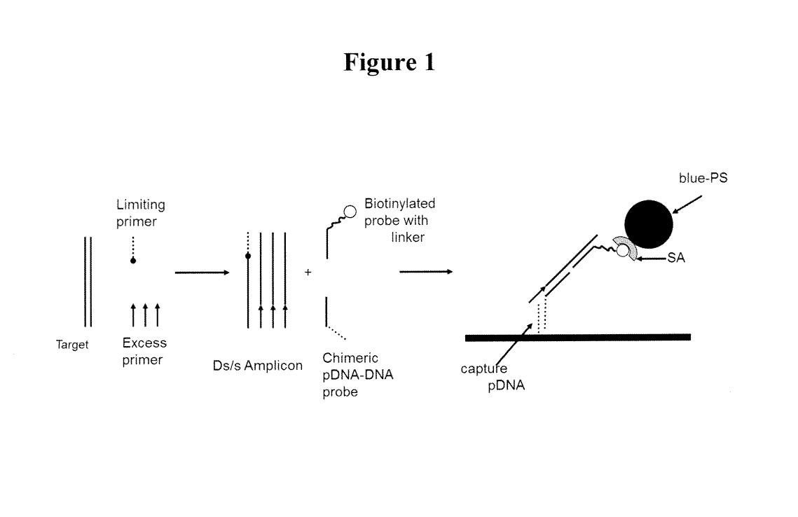

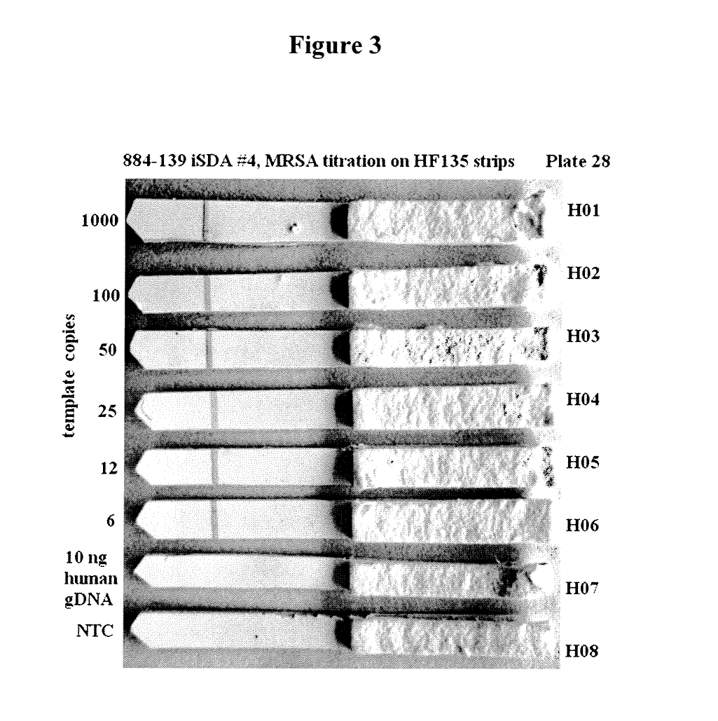

A similar iSDA amplification was performed except that probe 3 was replaced with probes 6 and 7 that allow capture and detection in a lateral flow format, as schematically depicted in FIG. 1, with the results shown in FIG. 3. Once the iSDA reaction was complete, 2 μL of the product was aliquoted into a well containing a streptavidin-coated label and a volume of buffer for running the lateral flow assay on HF135 nitrocellulose (Millipore), then the lateral flow strip was added to the well. In one example, 2 μL of the iSDA ldh1 reaction mixture was diluted in 100 μL of lateral flow buffer with the formulation 15 mM HEPES (pH 8), 1% Triton X-100, 0.5% BSA, 400 mM NaCl, 0.05% NaN₃, and 100 ng/μL streptavidin-coated 300 nm diameter blue-dyed polystyrene nanoparticles (Seradyn). To the diluted product was then added a nitrocellulose strip, 4×25 mm, containing an immobilized pDNA oligo complementary to the pDNA capture probe 6. The pDNA was immobilized via a cross-linked polythymidine tail at a concentration of 120 pmol/cm and a line width of approximately 1 mm. Positive results were visualized easily by the naked eye (as seen in FIG. 3).

### Example 2

This example illustrates the versatility of the design of primers from mecA gene sequences to allow iSDA amplification without denaturation. Nucleic acid was easyMag extracted from cultured S. aureus subsp. aureus COL. The primer, bumper and probe sequences of Design 1 and 2 are shown below in Table 2. The pDNA sequence is shown in brackets.

Table 2 below shows Designs 1 and 2 oligonucleotide sequences for mecA amplifications. Underlined sequences represent the nicking site for Nt.BbvC1B, the upper case sequence is mecA specific, the 5′-end lower case sequence is non-complementary to the mecA target, the pDNA sequence is shown in brackets, A* is Super A (U.S. Pat. No. 7,045,610), and Q14 is a hexaethylene glycol linker.

TABLE 2

| | | |
|---|---|---|
| Seq ID # | Description | Oligonucotide sequence |

Design

| | | |
|---|---|---|
| 8 | Forward | gaaacaatgtacctgtcacctcagcGACCGAAACAATGTGGAAT |
| | Primer | |
| 9 | Reverse | ttcaatagtcagttacttcctcagcGGAACGATGCCTAATCTCA |
| | Primer | |
| 10 | Probe | MGB-FAM-CCAATACAGGAACACAT-EDQ |
| 11 | Forward | GAAAATTTAAAATCAGAACGTGG |
| | Bumper | |
| 12 | Reverse | GCTTTA*TAATCTTTTTTAGATAC |
| | Bumper | |
| 13 | pDNA | [TTTTTTTTC]-(Q14-CAATGTGGA*ATTGG |
| | Capture | |
| | Probe | |
| 14 | Biotinilated | CCAATACAGGAACACAT-biotin |
| | Detection | |
| | Probe | |

Design 2

| | | |
|---|---|---|
| 15 | Forward | ccattatactacctgtctcctcagcGGCAAAGATATTCAACTAAC |
| | Primer | |
| 16 | Reverse | tagaatagtcagttacttcctcagcGCCATAATCATTTTTCATGTTG |
| | Primer | |
| 17 | Probe | MGB-FAM-CTTTTGAACTTTAGCATC-EDQ |
| 18 | Forward | GATAATAGCAATACAATCGCACA |
| | Bumper | |
| 19 | Reverse | GTGCTAATAATTCACCTGTTTGA |
| | Bumper | |
| 20 | pDNA | [CAAGAATC]-(Q14)-CTTTAGCATCAATAGTTAG |
| | Capture | |
| | Probe | |
| 21 | Biotinilated | GTTA*TAAATA*CTCTTTTGA-biotin |
| | Detection | |
| | Probe | |

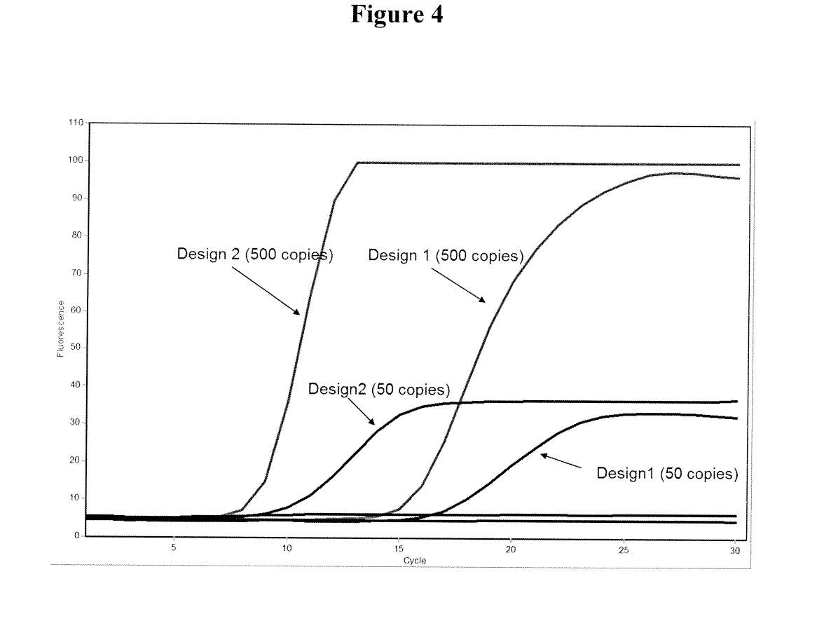

Using primers, probe and bumper oligonucleotides (Design 1, Seq. ID#8-12 and Design 2, Seq. ID #15-18) in the same way described in Example 2, efficient real-time iSDA was achieved as shown in FIG. 4.

### Example 3

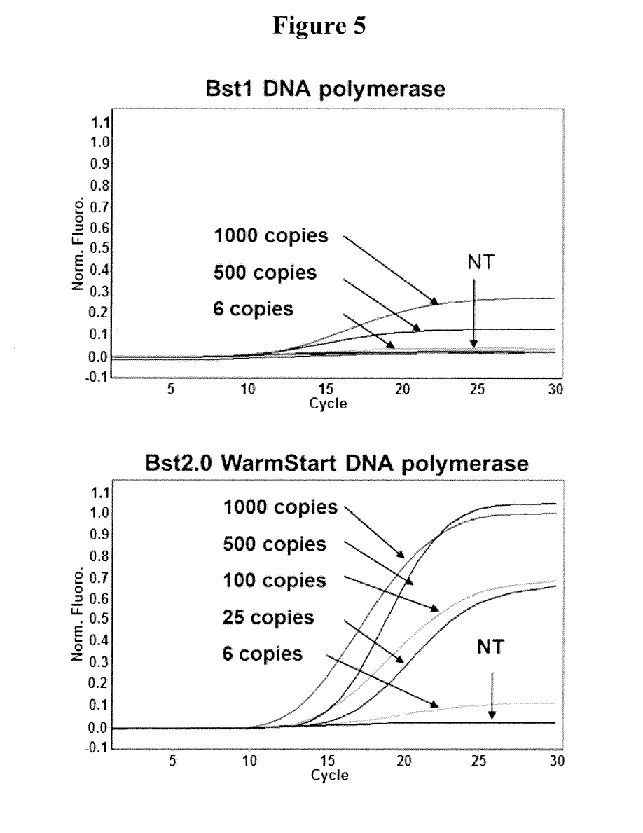

This example demonstrates the use of different polymerases in the real-time iSDA amplification. iSDA amplification was performed as described above using either Bst DNA Polymerase (portion of Bacillus stearothermophilus DNA Polymerase, New England BioLabs Inc., Ipswich, Mass.) or Bst2.0 WarmStart (an in silico designed homologue of Bacillus stearothermophilus DNA Polymerase I, New England BioLabs Inc.). The latter enzyme amplified mecA target and is active above 45° C. The results are shown in FIG. 5, indicating better performance with the Bst2.0 WarmStart enzyme.

### Example 4

This example demonstrates that although the Nt.Alw1 nicking enzyme successfully cut a PCR amplicon into which the NtAlw1 nicking site was designed, it did not cut extracted genomic DNA even though the ldh1 gene contains a natural nicking site for NtAlw1.

The sequences below in Table 3 were used to incorporate a nicking site into a PCR amplicon. The ldh1 specific sequences were designed with traditional PCR design software.

In Table 3 below, Design 3 and 4 oligonucleotide sequences for ldh1 amplifications were generated with the Eclipse Design Software 2.3. Underlined sequences represent the nicking site for NtAlw1, the upper case sequence is ldh1 specific, and the 5′-end lower case sequence is non-complementary to the ldh1 target.

TABLE 3

| | | |
|---|---|---|
| Seq ID # | Description | Oligonucleotide sequence |

Design 3

| | | |
|---|---|---|
| 22 | Limiting | aataaatcataaggatcAACGTGT |
| | primer-L1 | TATAGGTTCTGGTACA |
| 23 | Excess | aataaatcataaggatcTGAGCAT |
| | primer-E1 | CGACGCTACGTG |
| 24 | Forward | ATGGAAATTCTCTGGT |
| | Bumper1 | |
| 25 | Reverse | TGTC ACCATGTTCAC |
| | Bumper1 | |

Design 4

| | | |
|---|---|---|
| 26 | Limiting | aataaatcataaggatcTGGTGAA |
| | primer-L2 | CATGGTGACACTGAAT |
| 27 | Excess | aataaatcataaggatcGCCCTCA |
| | Primer E2 | GGACGTTGTTCAAG |
| 28 | Forward | AGCGTCGATGCTCA |
| | Bumper2 | |
| 29 | Reverse | AATTTGTTCAATTTGCG |
| | Bumper2 | |

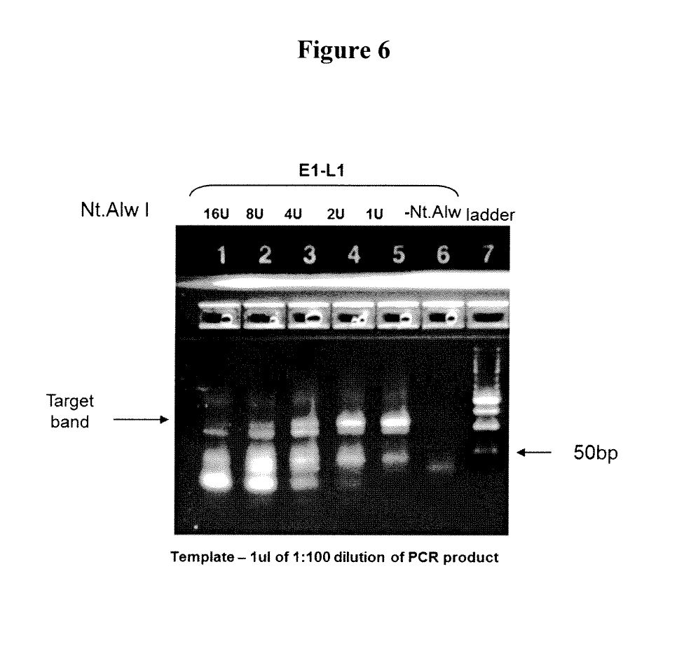

Primers of Design 3 and Design 4 were used to generate PCR amplicons which contain a nicking site for NtAlw1, yielding a convenient target containing a nicking site for NtAlw1. iSDA with the PCR-generated amplicon was analyzed on an agarose gel and the results are shown in FIG. 6.

### Example 5

This example illustrates the iSDA bi-plexing of ldh1 and an internal control (“IC”). The IC template contains nonsense, non-specific target DNA fragment in a plasmid vector. Preferably, the control nucleic acid comprises the sequence shown in Table 4 below.

In Table 4 below, oligonucleotide sequences for the amplification of the IC were generated as described above for iSDA amplification. Underlined sequences represent the nicking site for Nt.BbvC1B, the upper case sequence is IC-specific, and the 5′-end lower case sequence is non-complementary to the IC target. The same ldh1 primers, bumper, capture and detection oligonucleotides (Seq. ID#), 2 4-7, Table 1) were used for the bi-plexing of the ldh1 with the IC. The IC primers, bumpers, capture and detection probes sequences are shown in Table 4.

TABLE 4

| Seq ID # | Description | Oligonucleotide sequence |
|---|---|---|
| 30 | Limiting | ccaatatagtaacagtctcctcagcATTCGCCCTTCTGCACG |
| | primer-L1 | |
| 31 | Excess | ttcaaaagacccatacttcctcagCCTTCTCATTTTTTCTACCG |
| | primer-E1 | |
| 32 | Forward | TCGGATCCACTAGTAAC |
| | Bumper1 | |
| 33 | Reverse | GTGATGGATATCTGCAGAAT |
| | Bumper1 | |
| 34 | Chimeric | [ACATCACA]-Q14-GATCTTGTACCAATGC |
| | pDNA/DNA | |
| 35 | Biotinilated | CGTGGTCCGTAAAG-biotin TEG |
| | probe | |
| 36 | IC2 | TTTCACACAGGAAACAGCTATGACCATGATTACGCCAAGCTATTTAG |
| | | GTGACACTATAGAATACTCAAGCTATGCATCAAGCTTGGTACCGAGC |
| | | TCGGATCCACTAGTAACGGCCGCCAGTGTGCTGGAATTCGCCCTTCT |
| | | GCACGGACCAGTTACTTTACGGACCACGTACCGCATTGGTACAAGATC |
| | | TCCGGTAGAAAAAATGAGAAGGGCGAATTCTGCAGATATCCATCACA |
| | | CTG |

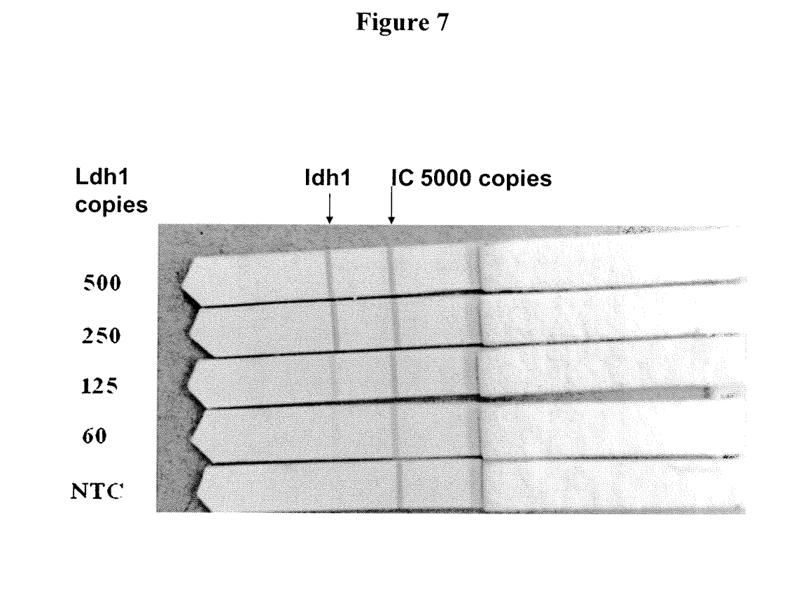

iSDA amplification was performed as described above except that the concentration for both ldh1 and the IC primers were 250 nM for the limiting primer and 500 nM for excess primer, forward and reverse bumper primers were at 50 nM, the chimeric pDNA-DNA probe and biotinylated probe at 200 nM each. Each target dilution contained 5000 IC2 copies. The amplification reaction was incubated at 48° C. for 30 minutes then it was analyzed by lateral flow analysis as described above. The lateral flow analysis is shown in FIG. 7 indicating for this particular assay a lower detection limit of 60 copies.

### Example 6

This example illustrates the probe specific iSDA detection and differentiation of S. aureus (BAA-1556, ATCC) and S. epidermidis (12228, ATCC).

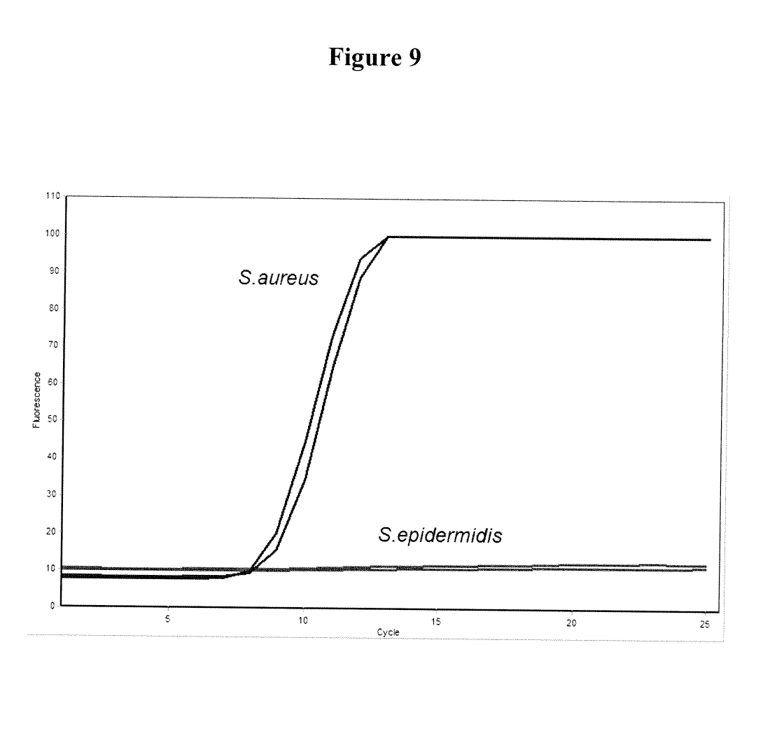

Cultures of S. aureus and S. epidermidis (5×10⁸ cfu/mL) were sonicated for 10 min in the waterbath sonicator (Branson 5510, Bransonic) The crude lysates were assayed for the ldh1 gene according to the method described in Example 1 at a concentration of 5×10⁴ cfu/reaction. Efficient specific detection of the ldh1 gene in S. aureus only is shown in FIG. 9.

### Example 7

This example illustrates the iSDA amplification of nucleic acid from the same sample extracted with different methods.

A S. aureus sample was extracted using the following extraction methods:
a) Extraction with chaotropic salts (8M guanidinium HCl or 4M guanidinium thiocyanate), with and without the silica spin column.

Bacterial cells (5×10⁸ cfu) were extracted according to the procedure described in Molecular Cloning: a laboratory manual. (pages 7-19, 7-24). DNA from each extraction was resuspended in 2004 of the TE buffer and divided into two 1004 aliquots. One aliquot was set aside for PCR and iSDA analysis, and another one was further purified on QIAmp DNA Mini Kit (Qiagen) spin columns according to the product manual. DNA was eluted in 100 μL of the elution buffer.
b) Phenol/chloroform extraction followed by ethanol precipitation. (Molecular Cloning: a laboratory manual, App.E3-E4).
c) Sonicat on for 10 min in the waterbath sonicator (Branson 5510, Bransonic).
d) 10% final concentration of Triton X100 incubation at room temperature followed by ethanol precipitation.

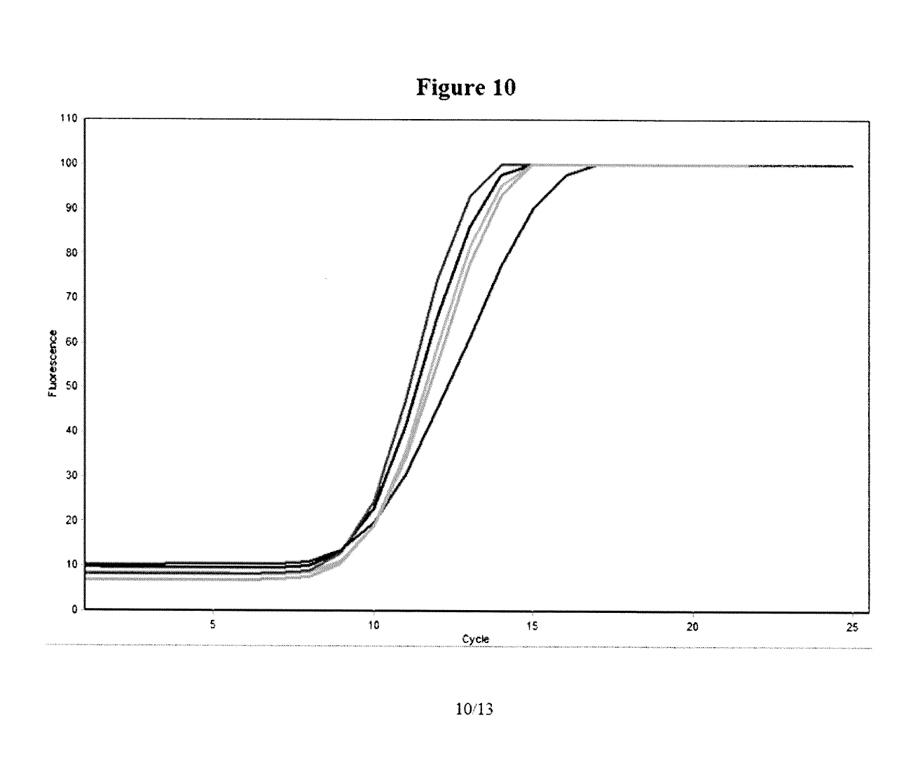

The concentrations of different non-denatured DNA nucleic acid fractions were normalized at 500 copies/reaction by real-time ldh1 PCR assay (described in U.S. patent application Ser. No. 13/479,557). As shown in FIG. 10, all five extractions gave essentially the same signal result at around cycle 9 (9 min). The NTC showed no amplification and is not shown.

### Example 8

This example illustrates the iSDA amplification of the ldh1 gene with primers and probes designed with the current disclosure in comparison with traditional designed primers and probes shown in Table 5

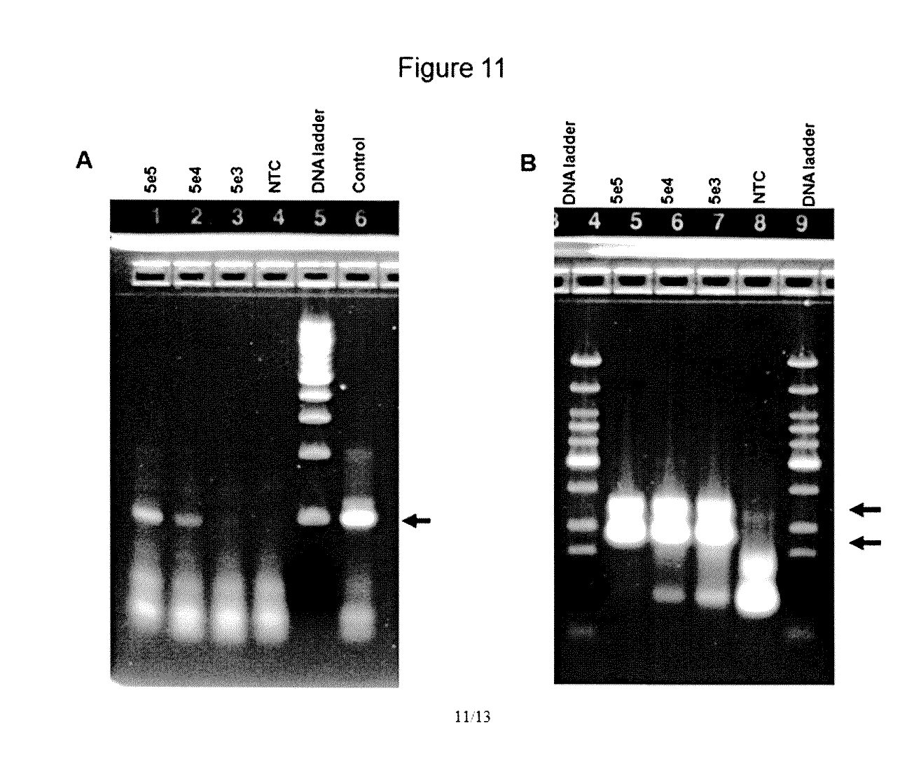

Using the method described in Example 1, the primers and bumper primers for the ldh1 gene described in Tables 1 and 5 were tested in which both sets of primers had target concentrations ranging from 5×10³ to 5×10⁵ target copies/reaction. The amplification reactions were analyzed by agarose gel electrophoresis as shown in FIGS. 11A and B. The arrows in FIGS. 11A and B refer to the amplicon products of amplification. As shown the amplification with the primers of the current disclosure showed substantial amplification at all three concentrations, while the conventional designed primers showed poor amplification FIG. 11A.

TABLE 5

| Seq ID # | Description | Oligonucleotide sequence |
|---|---|---|
| 37 | Limiting | gcattatagtacctgtctcctcagc |
| | primer-L1 | TGGTGACATGGTGACACTGAAT |
| 38 | Excess | ttgaatagtcggttacttcctcagc |
| | primer-E1 | GCCCTCAGGACGTTGTTCAAG |
| 39 | Forward | AGCGTCGATGCTCA |
| | Bumper1 | |
| 40 | Reverse | AATTTGTTCAATTTGCG |
| | Bumper1 | |

### Example 9

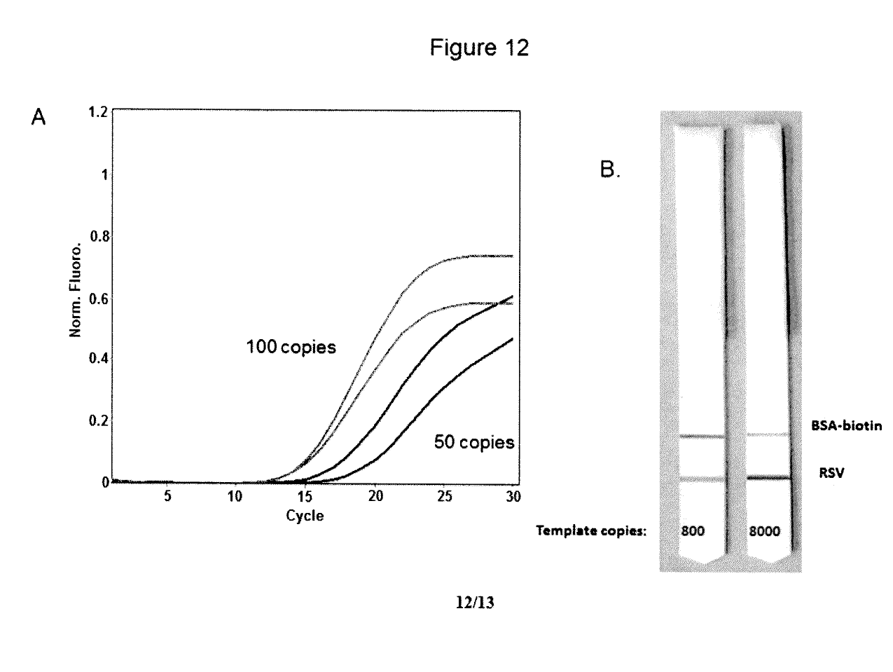

This example illustrates the one step RT-iSDA amplification of RSV nucleic acid. RT-iSDA uses the same final concentrations as disclosed for iSDA in [0049], except that 8U WarmStart Bst Polymerase was substituted for Bst Polymerase, 8U Nt.BbvC1 nicking enzyme was used per 10 μL reaction. In addition the reaction mixture contains 10U RNA inhibitor (Life Technologies), 0.5 μL Omniscript Reverse Transcriptase (Qiagne), template RNA and 1 μg BSA per 10 μL/reaction. Reaction mixture was followed in real-time for 25 minutes at 49° C. as illustrated in FIG. 12a) and lateral flow detection in FIG. 12b). Primers, bumper primers and probes are shown in Table 6 below. T*=Super T and other abbreviations have been described above. The lateral flow membrane has a test line of pDNA (immobilized by cross-linked polythymidine tail) and a BSA-biotin line as flow control.

TABLE 6

| Seq ID # | Description | Oligonucleotide sequence |
|---|---|---|
| 41 | Limiting | gcattatagtacctgtctcctcagc |
| | primer-L1 | GAATTCCCTGCATCAATAC |
| 42 | Excess | gcattatggtacctctctcctcagc |
| | primer-E1 | TA*TGTCA*ATATCT*T*CATC |
| 43 | Forward | AACTAAGGCCAAAGCTTATAC |
| | Bumper1 | |
| 44 | Reverse | CAGTCAGTAGTAGACCATG |
| | Bumper1 | |
| 45 | Chimeric | [TTTTTTTTC]-(Q14)-CTACAAA |
| | pDNA/DNA | TTATCACTTTGA |
| 46 | Biotinilated | TA*ATCGCATATTAACAG-biotin |
| | probe | TEG |
| 47 | FAM probe | MGB-FAM-TAATCGCATAT*T*AAC |
| | | AG-EDQ |

### Example 10

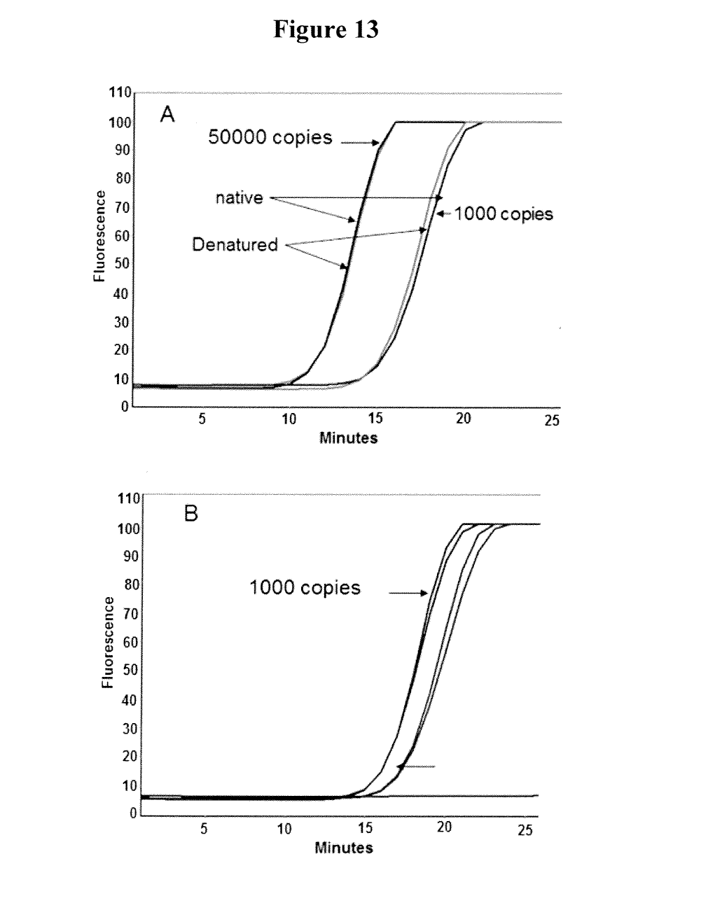

This example illustrates the iSDA amplification of native and denatured P. falciparum genomic DNA. Primers and probes were designed using mitochondrial DNA (Polley et. al., J. Clin. Microbiol, 48:2866-2871 (2010)) as a target and is shown in Table 7 below. Extraction from Plasmodium falciparum, strain NF54 and iSDA amplification were performed as described above. FIG. 13A shows identical real-time iSDA amplification for native and denature DNA at 95° C. for 5 minutes. FIG. 13B shows the amplification of native DNA at 100 and 1000 copies.

TABLE 7

| Seq ID # | Description | Oligonucleotide sequence |
|---|---|---|
| 48 | Limiting | gaatagacccatacatcctcagcGA |
| | primer-L1 | CTTGAGTAATGATAAATTGATAG |
| 49 | Excess | gaatagacccatacatcctcagcGA |
| | primer-E1 | CTTGAGTAATGATAAATTGATAG |
| 50 | Forward | CCA*CTTGCTTATAACTGTATG |
| | Bumper1 | |
| 51 | Reverse | GTTTCCA*TAGAAACCTTCAT |
| | Bumper1 | |
| 52 | FAM | MGB-FAM-ATTGATTCCGTTTTGAC- |
| | probe | EDQ |

### REFERENCES CITED

The following documents and publications are hereby incorporated by reference.

### U.S. and Foreign Patent Documents

U.S. Pat. No. 5,824,796
U.S. Pat. No. 5,912,340
U.S. Pat. No. 5,455,166
U.S. Pat. No. 7,282,328
U.S. Pat. No. 4,943,522
U.S. Pat. No. 7,488,578
US Application Publication No. 2012-0015358 A
U.S. Pat. No. 5,624,825
U.S. RE39885
U.S. Pat. No. 5,455,166
U.S. Pat. No. 5,422,252
U.S. Pat. No. 5,624,825
U.S. Pat. No. 5,712,124
U.S. Pat. No. 5,270,184
U.S. Pat. No. 5,470,723
U.S. Pat. No. 5,561,944
U.S. Pat. No. 5,736,365
U.S. Pat. No. 6,127,121
U.S. Pat. No. 6,660,845
U.S. Pat. No. 6,683,173
U.S. Pat. No. 7,045,610
U.S. Pat. No. 7,751,982
U.S. Pat. No. 7,799,554
U.S. Pat. No. 8,202,972
U.S. Patent Application Publication No. 2010/057862
U.S. Patent Application Publication No. 2011/0171649
U.S. Patent Application Publication No. 2012/0244535
PCT Publication WO 01/38584
PCT Publication WO 01/64958

### Non-Patent References

Afonina et al., Single Nucleotide Polymorphism Detection with fluorescent MGB Eclipse Systems in A-Z of Quantitative PCR, Ed. S. A. Bustin, International University Line, La Jolla, Calif., pages 718-731 and XII-XIII, 2004)
Besnier and Kong, EMBO Reports, 21: 782-786 (2001)
Ehses et al., J. Biochem. Biophys. Methods. 63:170-86 (2005).
Eschenmoser et al, Helvetica Chimica Acta, 76: 2161-2183 (1993)
Fran-Kamentskii. Artificial DNA; PNA & XNA, 2:1, 1-3 (2011)
Molecular Cloning: a laboratory manual
Niemz et al., Trends in Biotechnol., 29:240-250 (2011))
Nuovo, Diagn Mol. Pathol. 9(4):195-202 (2000)
M Panaccio and A Lew. PCR based diagnosis in the presence of 8% (v/v) blood. Nucleic Acids Res., 19: 1151 (1991)
Polley et al, J. Clin. Microbiol, 48:2866-2871 (2010)
Ramirez et al, Nucl. Acids Res. 40:5560-8 (2012)
Roberts et al., Nucl. Acids Res. 31: 418-420 (2003)
Walker et al., NAR 20: 1691-1695 (1992)
Walker, PCR Methods and Applications, 3: 1-6 (1993)
Walker et al., NAR 22: 2670 (1994)
Walker et al., Clin. Chem., 42: 9-13 (1996)
Walker et al., Clin. Chem., 42: 1604-8 (1996)
Walker et al., NAR 24:349 (1996)
Wang et al., Clin. Chem., 49: 1599 (2003)
White, Handler and Smith, Principles of Biochemistry 5^{th} Edition, McGraw-Hill Kogakush, Ltd, pages 192-197, 1993
Xu et al, PNAS 98: 12990-12995 (2001)
Zheleznaya et al., Biochemistry (Mosc). 74:1457-66 (2009)

What is claimed is:

1. An isothermal strand displacement amplification method, the method comprising:
(a) contacting a genomic nucleic acid having a target nucleic acid sequence with an amplification reaction mixture comprising:
a forward primer and a reverse primer,
wherein the forward primer has the formula:
A-B,
wherein B comprises a portion of the forward primer that is complementary to the target nucleic acid sequence, and wherein A comprises a portion of the forward primer that is non-complementary to the target nucleic acid sequence and comprises a forward nicking enzyme recognition sequence,
wherein the reverse primer has the formula:
A′-B′,
wherein B′ comprises a portion of the reverse primer that is complementary to the target nucleic acid sequence, and wherein A′ comprises a portion of the reverse primer that is non-complementary to the target nucleic acid sequence and comprises a reverse nicking enzyme recognition sequence, and
wherein the forward primer and the reverse primer comprise sequences optimized by software for specific hybridization and efficient elongation,
a polymerase enzyme having strand displacement activity, and
a nicking enzyme specific for the nicking enzyme recognition sequence;
(b) incubating the amplification reaction mixture and the genomic nucleic acid under amplification conditions suitable for amplification of the target nucleic acid to produce an amplified target nucleic acid,
wherein the contacting step and the incubating step are carried out at a temperature between about 40° C. and about 65° C. and amplification of the target nucleic acid occurs without thermal denaturation; and
(c) detecting the amplified target nucleic acid.

2. The method of claim 1 wherein the amplification reaction mixture further comprises one or more bumper oligonucleotides.

3. The method of claim 1 wherein the genomic nucleic acid is RNA and wherein the amplification reaction mixture further comprises a reverse transcriptase enzyme.

4. The method of claim 1 wherein the genomic nucleic acid is double stranded.

5. The method of claim 1 wherein the target nucleic acid sequence is single stranded.

6. The method of claim 1 wherein the step of detecting the amplified target nucleic acid comprises using fluorescence resonance energy (FRET), radiolabels, lateral flow, or enzyme labels.

7. The method of claim 1 wherein the step of detecting the amplified target nucleic acid comprises hybridizing an oligonucleotide probe to at least a portion of the amplified target nucleic acid.

8. The method of claim 6 wherein the oligonucleotide probe is a fluorescent generation probe.

9. The method of claim 7 wherein the oligonucleotide probe comprises a minor groove binder (MGB), a fluorophore, and a quencher.

10. The method of claim 7 wherein the oligonucleotide probe is a FRET probe.

11. The method of claim 7 wherein the oligonucleotide probe fluoresces when hybridization to the amplified target nucleic acid occurs.

12. The method of claim 7 wherein the oligonucleotide probe is cleaved to produce a fluorescent signal.

13. The method of claim 1 wherein the step of detecting the amplified target nucleic acid comprises using lateral flow.

14. The method of claim 1 wherein at least one of the forward primer and reverse primer comprises a fluorescent label.

15. The method of claim 1 wherein the step of detecting the amplified target nucleic acid comprises attaching the amplified target nucleic acid to a solid support and detecting the amplified target nucleic acid with an oligonucleotide probe having a fluorescent label.

16. The method of claim 1 wherein the amplification reaction mixture further comprises an internal control.

17. The method of claim 1 wherein the software comprises the Vienna Folding Package.

18. The method of claim 1 wherein the software comprises software for adjusting the Tₘ of the forward primer and the reverse primer by calculating duplex stabilities using an algorithm applying a nearest-neighbor model for duplex formation thermodynamics for each neighboring base pair.

19. The method of claim 1 wherein the forward primer and the reverse primer are present in different concentrations in the amplification reaction mixture.

20. The method of claim 1 wherein at least one of the forward primer and reverse primer is substituted with at least one modified base.

21. The method of claim 1 wherein at least one of the forward nicking enzyme recognition sequence and the reverse nicking enzyme recognition sequence comprises a cleavage site for Endonuclease V.

22. The method of claim 1 wherein the contacting step and the incubating step are carried out at a temperature between about 45° C. and about 55° C.

23. A method of claim 1 wherein A′ comprises a sequence for a cleavage site.
